(19) United States
(12) Patent Application Publication
(10) Pub. No.: US 2026/0262148 A1
(43) Pub. Date: Sep. 3, 2026

(54) LED LIGHTING DEVICE ABLE TO KILL CORONAVIRUS

(71) Applicants: Herman DIAZ ARIAS, ATIZAPAN DE ZARAGOZA (MX); Ma. Isabel De Jesus PIER ROMERO, ATIZAPAN DE ZARAGOZA (MX)

(72) Inventors: Herman DIAZ ARIAS, ATIZAPAN DE ZARAGOZA (MX); Ma. Isabel De Jesus PIER ROMERO, ATIZAPAN DE ZARAGOZA (MX)

(21) Appl. No.: 18/867,022

(22) PCT Filed: May 18, 2023
(86) PCT No.: PCT/IB2023/055143

§ 371 Date: Nov. 19, 2024

(30) Foreign Application Priority Data
May 20, 2022 (MX) MX/A/2022/006193

(51) Int. Cl. H05B 45/20; A61L 2/084; A61L 9/18; H05B 45/325; H05B 47/16; H05B 47/175

(52) U.S. Cl. CPC H05B 45/20; A61L 2/084; A61L 9/18; H05B 45/325; H05B 47/16; H05B 47/175

(57) ABSTRACT

The device to give LED lighting lamps capacity for coronavirus destruction, allows to give any white light LED luminaire or LED lighting lamp an anti-coronavirus sanitizing capacity through an electronic circuit that drives white lighting LEDs, RGB or fluorescence type in an unconventional way, producing intense and very brief pulses of light within the 400 to 460 nm band, which allows the LED luminaries to produce white light with very narrow pulses of light with 400 to 460 nanometers wavelength controllable using a wall switch. The destruction of coronaviruses is done by weakening the lipid cover of coronaviruses and works for all coronavirus variants.

### FIELD OF THE INVENTION

The present invention is developed in the fields of optoelectronics, electronics and physics.

### BACKGROUND OF THE INVENTION

Since the middle of the last century, light radiation equipment has been used for sanitation purposes, especially in bactericidal applications, and this was done through the use of ultraviolet light, which due to its characteristics we should call ultraviolet radiation rather than ultraviolet light, since this type of radiation is invisible to the eye. with the appearance of the COVID 19 pandemic, the need to use various means to combat the spread of coronaviruses has been seen, the use of ultraviolet radiation, has shown serious side effects such as the induction of mutations, structural deterioration effects on plastic surfaces and carcinogenic effects in humans and animals, however it has recently been discovered that visible light within a certain wavelength, (400 to 460 nanometers) which is a blue light, has the property of effectively attacking coronaviruses, since this light weakens the lipid covering that protects the center of coronaviruses where their genetic material is located, (study in this regard carried out by the Scientific Department of the Army Medical Center, Rome, Italy and the one carried out by researchers from the Mount Sinai School of Medicine, NY, USA) in addition to the fact that on this lipid covering, there is the set of proteins that allows coronavirus to enter the cells it uses to reproduce. Light radiation with a wavelength of between 400 and 460 nanometers has been seen to not only destroy coronaviruses, but also to do so without any of the drawbacks that ultraviolet radiation usually presents.

Although it is possible to incorporate this type of blue lighting into the luminaires used in the lighting of houses, shops and factories, it is not desirable to give a blue hue to the lighting, since in addition to being very annoying, it directly affects circadian cycles, the effect of blue light in the waking state has been studied extensively in alert and work situation of human beings.

A previous precedent on the use of blue light pulses for the destruction of the coronavirus by weakening its lipid cover is present in patent application MX/a/2020/004121 even prior to the international studies that corroborate it, which has a clinical use approach where blue light pulses of between 400 and 460 nanometers combined with ultrasound are used to treat patients infected with COVID is also present in the patent application MX/a/2020/014241 which is focused on the sanitization of areas or objects with pure blue light and ultrasound as well as for the manufacture of non-passive masks, however in these cases the equipment described in said patents is focused on the use of blue light exclusively and in no way raises the possibility of creating or adapting white light illumination LED emitters that could have the same capacity for viral destruction while maintaining its lighting properties.

In accordance with the above, we saw the need to design a type of lighting system that has an appearance as close as possible to white light lighting but that could incorporate within this light, a higher percentage of blue radiation of between 400 to 460 nanometers with the necessary characteristics for a rapid and continuous destruction of coronaviruses. This makes it possible to greatly reduce the proliferation of coronaviruses, as well as current and future variations of them, since unlike vaccines, this process does not need to be specifically designed and modeled to act on a certain strain of coronavirus, the action of pulses of 400 to 460 nm of high intensity is enough for destroy any generic coronavirus.

For this, it is necessary to generate and deliver a sufficient amount of blue light energy that does not turn the white light luminaires into blue light luminaires, but their appearance must be basically white light, this may allow sustained irradiation, which will go unnoticed and which will destroy coronaviruses both in the air and on exposed surfaces.

### BRIEF STATEMENT OF THE INVENTION

The technology that is the subject of the present invention described here is intended for the destruction of the coronavirus and consists of a circuit that manages the energy provided to the two most generalized types of white lighting with LEDs, adding to these the possibility of having, in addition to the lighting capacity of neutral white light, with a virucidal effect that can destroy coronavirus, the two types of traditional LED lighting to which we refer are the RGB system, which uses a set of LED emitting diodes with blue, green and red colors that when mixed with their lights give rise to practically white light illumination, the second most generalized type of LED white light uses one or more blue LEDs inside a cavity that has a fluorescent screen or filter, which converts blue radiation into an emanation of practically white light, this type of lighting system is called LED fluorescent lighting. The electronic circuits presented here allow an assembly that, when combined with fluorescent emission LEDs or RGB white light illuminators, generates a series of overexcitation voltage pulses (voltages of 1.5 or more times the nominal working voltage of the LEDs or LED arrays), which in turn allows the LED lighting systems to have a new property that effectively combats coronavirus without altering circadian cycles and without giving a noticeably bluish coloration to the lighting. The overexcitation module or circuit powers the LEDs in a completely different way than conventional LED drivers, since the LEDs are normally powered by a controlled current either constant or pulsed, but the LEDs in their conventional mode of operation, never exceed their maximum operating voltages, while our circuits imposes on the LEDs voltages that exceed several times the nominal operating voltage of the LEDs, but this is done for an extremely short time, usually a few microseconds in order to avoid the destruction of the light-emitting diodes, this procedure produces pulses of polychromatic light energy in addition to the base wavelength of a monochromatic LED, this special feature not only improves the performance of white LED luminaires, but also makes it possible to produce extraordinarily narrow and powerful pulses of blue light that enable the destruction of the coronavirus.

It is important to note that some of the circuits that we present for the assembly of sanitizing lighting system against coronavirus, have a remote control device that allows increasing or decreasing the relative intensity of the radiation between 400 and 460 nm imperceptibly with respect to the general emitted radiation of white light. This device can act by the simple action of wall switches normally used to turn the lighting of a sector on or off.

The circuits described below can be used as a driver that can be easily incorporated into luminaires equipped with white LED emitters or can be integrated into units comprising both the emitters and the controls or drivers.

### BRIEF DESCRIPTION OF THE FIGURES

FIG. 1. shows the electronic circuit module with voltage overexcitation for the non-conventional driver for lighting LEDs that allows to convert an LED luminaire into a sanitizing lighting system capable of destroying coronaviruses whether they are RGB or fluorescence LED emitters.

FIG. 2. It shows the two different types of voltage overexcitation circuits for lighting LEDs, one with constant overvoltage pulses and one with relaxation or variable overvoltage pulses.

FIG. 3. It features a module with the electronic circuit for overexcitation of constant voltage pulses, applied to an array of white illumination LEDs by fluorescence, as well as emission level control in the band from 400 to 460 nm.

FIG. 4. Displays the operation of a control for the energy level in the 400 to 460 nm radiation emitted by using a wall switch or remote switch.

FIG. 5. It shows a sanitizing white light illumination device using fluorescent LED emitters and a relaxation voltage or variable overvoltage overexcitation module.

FIG. 6. It displays a modular sanitizing lighting circuit with constant voltage pulse overexcitation and proportional radiation remote control.

FIG. 7 This figure shows a comparison of the driver for RGB LEDs in a conventional manner and with overvoltage pulses.

### DETAILED DESCRIPTION OF THE INVENTION

The destructive effect that light with wavelengths within 400 to 460 nanometers has on all types of coronaviruses has already been proven because this kind of light radiation weakens the fatty or lipid coating that protects the genetic material of this type of virus, it is important to note that the technology we describe here is focused solely on the destruction of coronaviruses. What has been proven so far is that this portion of the light spectrum that goes from 400 to 460 nm destroys coronaviruses, but unless a very high level of energy is used, the destruction process can take a long time and for practical purposes, it is not convenient to use blue light emitters (400 to 460 nanometers) as sanitizing elements in parallel to the normal lighting that is a predominantly white light, since in addition to being very annoying the fact that everything illuminated would have a blue tint, this type of hue has negative effects on people's circadian cycles, in such a way that states of alertness or wakefulness could extend throug daylight hours when in certain places you no longer want to work but to rest, that is why we have designed a way to incorporate into a conventional white LED lighting system, the ability to radiate the illuminated area by the luminaires with strong pulses of light with wavelength of 400 to 460 nanometers but making these pulses very short in time but strong enough to generate the sanitizing effect without giving the luminaire a blue hue, taking advantage of the white LED illuminators without altering its generall lightning characteristics, for this, we use what we call overvoltage pulses, which consist of feeding the LED diodes with voltage pulses higher than their nominal level, for example, we can power or excite an LED whose operating voltage is 2 volts, with pulses of 40 volts but with a duration in the range of a few microseconds, these repetitive pulses are generated by circuits that prevent the destruction of the LEDs by overheating.

Next we describe an electronic circuit that we call a voltage overdrive module or circuit for white light lighting LEDs, which contains a driver or controller that manages the LED emitters through voltage pulse impacts with a level higher than 1.5 times the nominal operating voltage of the LEDs or LED arrays instead of using controlled current sources.

We use the term driver because of its widespread use to describe a circuit that powers and controls an assembly of LEDs.

The two basic white light lighting systems with LEDs are the so-called RGB emitters, which use LEDs of three different colors (Red, Green, Blue) whose light is mixed to form an apparently white light emission, and the so-called fluorescence emitters which use one or more blue LEDs, placed in a cavity behind a fluorescent screen which is excited by the blue radiation and produces white secondary light emissions, in both cases, what our design does is to excite the blue LEDs of the primary emission by means of pulses of very short duration in the range of microseconds but with an amplitude in voltage that exceeds in any case the nominal voltages of the LEDs or matrix arrays of blue LEDs, which consequently gives an emission of blue primary light that mostly covers the spectrum relative to the band of 400 to 460 nanometers, if the blue primary illumination to obtain white light were done in a conventional way using, for example, blue LEDs of 460 nm. the emission of primary blue light would only contain that wavelength, but when using the overvoltage pulses, secondary emissions are produced within the band that goes from 400 to 460 nm, which helps both in the sanitizing aspect and in the lighting performance, delivering a greater amount of blue light content without modifying the perception of the apparently white light. This device for converting white LED lighting lamps for the destruction of coronavirus, is basically constituted as described above by an LED emitter of white light and a module or exciter that controls the emitter by feeding it with pulses of short duration but of a voltage higher than the nominal voltage of the LED, being that this type of excitation can generate in combination with the white light emitter, a controlled amount of additional radiation in the range of 400 to 460 nanometers in the form of pulses that by their nature do not generate a bluish perception in illumination.

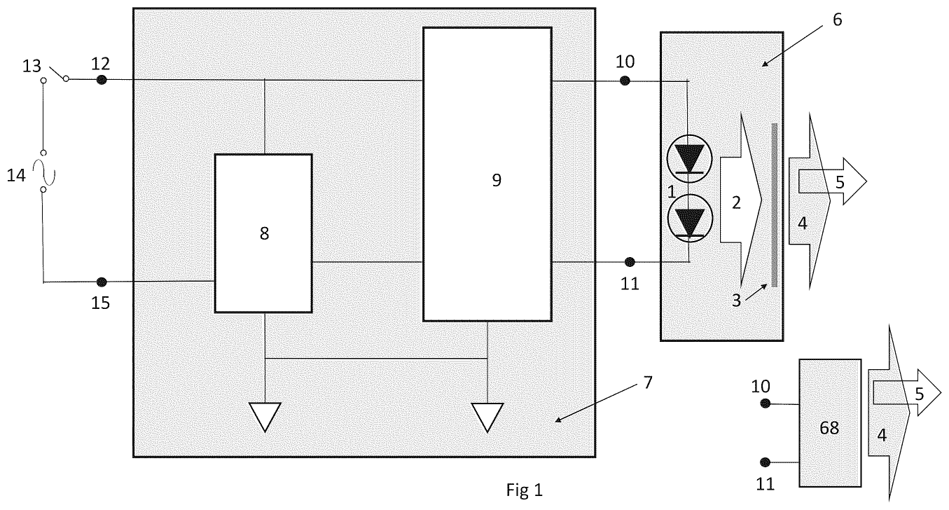

The device for give LED lighting lamps capacity for coronavirus destruction, basically consists as can be seen in FIG. 1 of a voltage overexcitation module (7) which is connected on one side to the alternating current electrical power supply system (14) by means of two terminals (12), (15) and a wall switch (13), which is usually the switch that controls the ambient lighting of a place or area, the overexcitation voltage module also has two additional outputs, the positive output terminal (10) and the negative output terminal (11) that are connected respectively to the corresponding inputs of the LED illuminator which can be a fluorescence white light LED illuminator (6) or an RGB white light illuminator (68), which will emit respectively mostly white light for illumination and an surplus of blue light sanitizing pulses (5) with wavelength from 400 to 460 nm.

Referring first to the assembly using fluorescent white light LED illuminators (6), the circuit of the voltage overexcitation module (7) acts by sending directly to the LED or array of blue LEDs that fluorescence white light emitters normally have inside, and which we will refer to hereinafter as LED emitter (1), short-duration (microseconds) overvoltage pulses with amplitude levels greater than 1.5 times the nominal excitation voltage of the LED or blue LED array, this in turn produces a primary emission of blue light radiation (2) which impacts the white light conversion screen of fluorescent material (3), with which an emission of white light for illumination (4) is obtained accompanied by a surplus of blue light sanitizing pulses (5), this is mainly due to the fact that fluorescent conversion screens have limited capacity to convert radiation in the blue band into white light, that is, this screen can perform the conversion up to a certain amount of blue radiation, but there comes a time when an excess of blue radiation can no longer be converted and will therefore pass through the screen retaining its characteristic wavelength of between 400 to 460 nm. and only a fraction of the emitted blue light will be converted to white light, allowing a portion of the microsecond light pulses, but very intense due to the overvoltage excitation, to pass through the screen so that the entire illuminator assembly generates the desired combination of white light and sanitizer blue light. The voltage overexcitation module (7) also has a remote sanitization level control (8) and a voltage overexcitation circuit or driver (9).

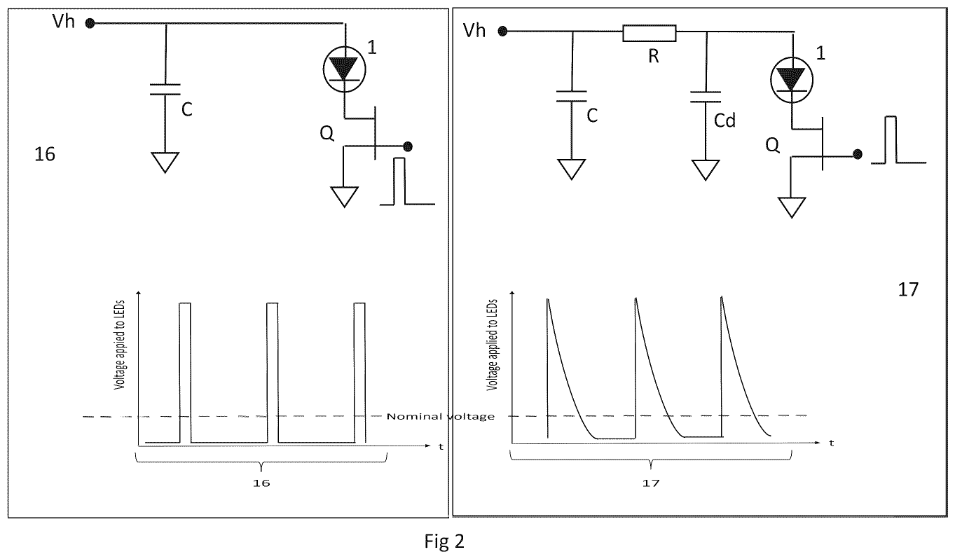

In FIG. 2 the two versions of the overexcitation circuit that we use for this purpose are schematically described, the diagram (16) shows the nature of the overexcitation pulses that are applied to the LED emitter (1) in the version of constant voltage pulses, which as can be seen is a voltage that exceeds its nominal voltage, in its simplest form, the circuit to perform this task, is formed by an energy storage capacitor (C) whose value fluctuates between 500 and 2000 microfarads, being that this capacitor is charged at a voltage Vh that exceeds the nominal operating voltage of the LED emitter (1), when a switch (Q) which in this case is a field effect transistor is put into complete conduction by means of a pulse in its gate, all the Vh voltage will be applied through the LED emitter (1), this is done for a few microseconds so that the amount of energy provided to the LED emitter (1) over the course of a second does not exceed the power rating of the LED emitter (1), this is determined by adding the energy of all the pulses that occur in a second avoiding the destruction of the LED emitter. As can be seen, the voltage through the LED emitter (1) will remain constant for the entire duration of the pulse.

The second version of the overexcitation system shown in diagram (17) of FIG. 2 is made up of a circuit similar to a relaxation oscillator, this version has in some cases the advantage of being a more economical and compact solution than the version of constant overvoltage pulses, in this case, a capacitor (C) with a value of between 500 and 2000 microfarads, is charged at a voltage Vh with which in turn a capacitor (Cd) of smaller proportions of between 1 nanofarad and 1 microfarad is charged through a resistor (R) and when the voltage through (Cd) reaches a certain value, it is discharged through the LED emitter (1) through the action of the electronic switch (Q) which in this case is a field effect transistor, this produces the effect that the voltage through the LED emitter (1) starts at a very high value and then this voltage decreases until the discharge flow through it is interrupted as can be seen in the diagram (17) and this is repeated successively taking care that the amount of energy applied in each pulse multiplied by the number of pulses emitted in a second, does not exceed the power rating of the LED emitter (1) as each LED or array of LEDs has a specific power rating.

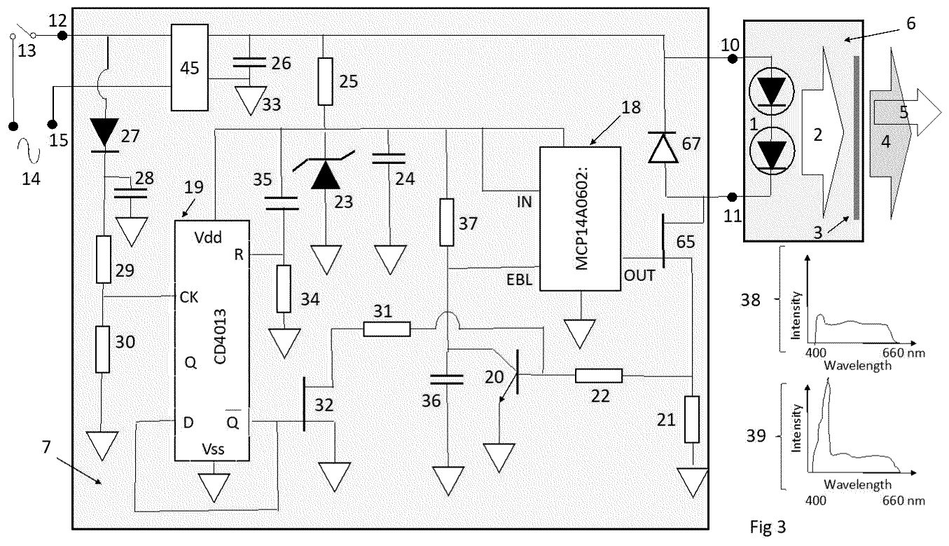

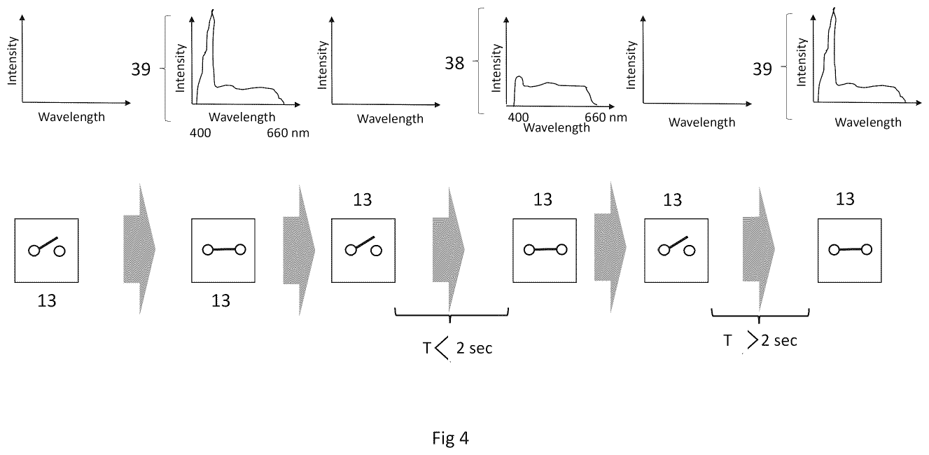

Fluorescence white light LED emitters are arguably the most widely used in industry, commerce, and home because they have significant advantages in terms of cost and ease of operation over RGB type white light LED emitters, although the light emission spectrum of fluorescence LED emitters covers a wide spectrum or a wide range of wavelengths that typically range from the lower 400 nanometers to the upper 600 nanometers, its sanitizing effect on coronaviruses is gradual and takes a long time of exposure for the coronaviruses to be destroyed, which is why our system employs a very specific process to strengthen the delivery of energy within the 400 to 460 nanometer band but with the possibility that this does not alter the white appearance of the lighting and without having a negative effect on people's circadian cycles, the latter is reinforced by the use of the conventional wall switches that are used to activate lighting in certain areas, as an element that allows the maximum amount of light energy within the blue band to be controlled in a simple way and at a minimum additional cost, in FIG. 3, an electronic diagram corresponding to a voltage overexcitation module (7) is presented, connected to a fluorescent white light LED illuminator (6), in this case the voltage overexcitation module (7) is equipped with a circuit that allows modifying the amount of light energy in the wavelength range from 400 to 460 nm. By means of the action of the wall switch (13), graphs (38) and (39) illustrate the light spectrum of emission controlled by the action of the wall switch (13) which operates as follows, if the user at a given time closes the wall switch (13), we will have a light emission similar to the one presented in the graph (39), and it will remain so until the user activates the wall switch (13) by turning off the lighting unit, but if the user, once he has turned on the lighting unit by means of the wall switch (13), turns it off and on again before a pre-set time (one to three seconds) has elapsed, the light emission will have a configuration similar to that shown in graph (38), considerably reducing the amount of energy emitted within the band from 400 to 460 nm., in this modality, the modulation system will continue to have a sanitizing effect on coronaviruses, but will present a minimum of interaction with the circadian cycles of the people who are in the illuminated enclosure, in FIG. 4, the sequence of operation of the wall switch (13) is graphically presented, which allows the percentages of irradiation to the illuminated area to be controlled.

In FIG. 3 a voltage overexcitation module (7) circuit is presented in the constant overvoltage pulse mode as shown in diagram (16) (see FIG. 2), in this case, the overexcitation driver circuit produces pulses of very short duration but with a voltage that exceeds the nominal value of the operating voltage of the LED emitter (1) to which said circuit is connected by means of the terminals (10), (11), this circuit is presented made by the use of certain integrated circuits but these can be replaced by integrated or discrete electronic elements that can perform the same function, simply by means of a cost-benefit evaluation criterion, thus the FLIP-FLOP circuit (19), which is a bistable multivibrator such as the CD 4013, can be replaced by another circuit that performs the same function or by a microcontroller that emulates the same function that the CD4013 does and the same applies to the MOS driver or current amplifier (18), which in this case is a MOS MCP 14A0602 driver, which can be replaced by any current amplifier circuit or a circuit dedicated to the operation of MOS transistor gates. When the wall switch (13) is closed, a rectifier/regulator circuit (45) in conjunction with capacitor (26) provides a direct current voltage whose voltage magnitude is greater than 1.5 times the nominal operating voltage of the LED emitter (1), while the rectifier/regulator circuit (45) and capacitor (26) will define the ground point (33), the active and passive operating circuits of the overexcitation driver are powered by a voltage from a regulator that allows to have a voltage at the level required by the operating integrated circuits and that will depend on the type of family of circuits to which they belong, for example for a CD4013 that belongs to the CMOS family, a voltage between 5 and 15 volts is appropriate as it will also be for the supply of the current amplifier (18), the diagram shows how this voltage can be obtained by the action of a Zener diode (23) fed by a resistor (25) and accompanied by a capacitor (24) as a damping and filtering element; if at any given time the voltage at the activation input (EBL) of the current amplifier (18) circuit or MOS driver, is at a logic level one equal to the input (IN), in this case the output (OUT) of the current amplifier (18), will be at a logical level one, very close to the supply voltage value determined by the Zener diode (23), this will cause the transistor (65), which can be a field-effect transistor, to enter full conduction, causing practically all the voltage accumulated in capacitor (26) to be presented on the terminals of the LED emitter (1) because the resistor (21) that connects the transistor (65) source with ground has a value of just a few tenths of an ohm (between 0.1 and 1 ohm), this resistor (21) operates as a monitoring element that allows calculating the amount of current that is flowing through the LED emitter (1), the voltage through resistor (21) is connected to the base of NPN transistor (20) through resistor (22) and if transistor (32) is not in conduction, when the voltage across the monitoring resistor (21) exceeds 0.7 volts, the NPN transistor (20) will enter conduction draining the charge from capacitor (36) which in turn determines that transistor (65) stops conducting terminating the voltage pulse on the LEDs, however if FET transistor (32) is activated, a voltage divider will be formed between resistor (22) and resistor (31), one of whose ends will now be practically at ground level, in this case, for NPN transistor (20) to be activated, a voltage level will be required through the monitoring resistor (21) greater than 0.7 volts, increasing the amount of energy of each pulse which determines that the amount of energy of each voltage pulse over the LEDs increased, once this has happened the NPN transistor (20) will also enter into cut-off since there will no longer be current flow through resistor (21), allowing capacitor (36) to charge through resistor (37) until it reaches the level that allows the activation of the current amplifier (18) again activating the transistor (65) again to initiate the generation of a new voltage pulse on LED emitter (1), a protection diode (67) helps protect the LED emitter from reverse voltage overthrows.

As can be seen, the voltage over resistor (21), allows to control the maximum amount of energy provided to the LED emitter (1) in each pulse, which is why the transistor (32) can act as an additional control element over this amount of energy since if this transistor (32) is in cut-off, the NPN transistor (20) will begin to conduct as soon as the voltage through resistor (21) exceeds 0.7 volts, but if transistor (32) is in full conduction, a voltage divider will be formed with resistor (22) and resistor (31) causing the voltage across resistor (21) necessary for NPN transistor (20) to start conduction increases depending on the relationship between resistors (22) and (31), in this case the amount of energy allowed in each pulse on the LED emitter (1) would be increased, the action of transistor (32) is controlled by the inverted output of the FLIP-FLOP circuit (19), so depending on the state that this output keeps, the emission of a greater or lesser amount of light within the band of 400 to 460 nanometers will be determined, the action of this FLIP-FLOP type circuit (19) is determined by a sequence of actions on the wall switch (13) operating as follows: when the wall switch (13) is activated for the first time, the capacitor (35) will be discharged and therefore will establish a reset condition on the FLIP-FLOP circuit (19), the clock signal for the FLIP-FLOP circuit (19) is given through its clock input connected to resistors (29), (30) and the damping capacitor (28) which in turn is connected by the diode (27) to the power supply input at terminal (12), this allows the signal to the clock input to always be within the operating limits of the clock, in this condition, the inverse output Q will be at a level one by driving transistor (32), if the wall switch (13) is turned off and reactivated within a couple of seconds, capacitor (35) will have already been charged to a zero level by the grounded resistor (34) and by entering a new clock signal into the FLIP-FLOP circuit (19), will change state because the data input is connected to the inverse Q output and now this output will pass to ground level cutting off the action of transistor (32) which directly reduce the amount of energy allowed in each pulse on the LED emitter (1), as can be seen, the amount of energy emitted in the range of 400 to 460 nanometers, can be controlled by a simple action of the wall switch (13) producing either a low sanitization light or a high sanitization light for coronavirus, the sequential operation of this control can be seen in FIG. 4 and it is important to establish that given the characteristics of the white light fluorescence LED emitters, which are designed to accept a certain amount of blue light energy that can be converted to white light within a specific range of wavelengths, and when these energy level and wavelength limits are exceeded, the surplus contributes little to the generation of more white light, so that some of the surplus excitation blue light passes through the fluorescent screen maintaining a mostly blue condition but given that in our design these pulses are generated intensely and briefly, they are not mostly appreciated by the user giving the general appearance that the illuminating light is basically white. FIG. 4 shows an operation sequence of the wall switch (13) where it can be seen that this control works as follows, when activating the wall switch (13) the sanitizing lighting system will be activated with a maximum fullness of radiation intensity in the band of 400 to 460 nanometers, this condition will remain all the time that the wall switch (13) remains closed but if at a given time the user wants to limit this amount of blue sanitizing emission, simply opening and closing wall switch (13) within 2 seconds, will cause the sanitizing lighting system to continue to have this effect but with a lower production of light pulses of 400 to 460 nanometers, this condition will prevail until the user decides to turn off the light, If you do this and more than 2 seconds pass before activating again, the lighting system will be activated the next time the wall switch (13) is closed, producing maximum sanitization against coronavirus, this faculty that our design presents, is very useful for example when the maximum sanitization level is being used in the room of a home but the user already wants to prepare to rest and sleep, many studies have revealed that if ambient lighting has a lot of wavelength participation in the blue band, it will be difficult to rest and fall asleep, since bluish lighting induces a state of alertness, however we want to note that our excitation system for white LED emitters, produces a minimal sensation of blue tint and has little effect on circadian cycles but we give the user the possibility of reducing the emission of blue radiation to a minimum even if the anti-COVID sanitization effect continues to be produced, although with less blue emission, it takes the lighting system more time to destroy coronaviruses.

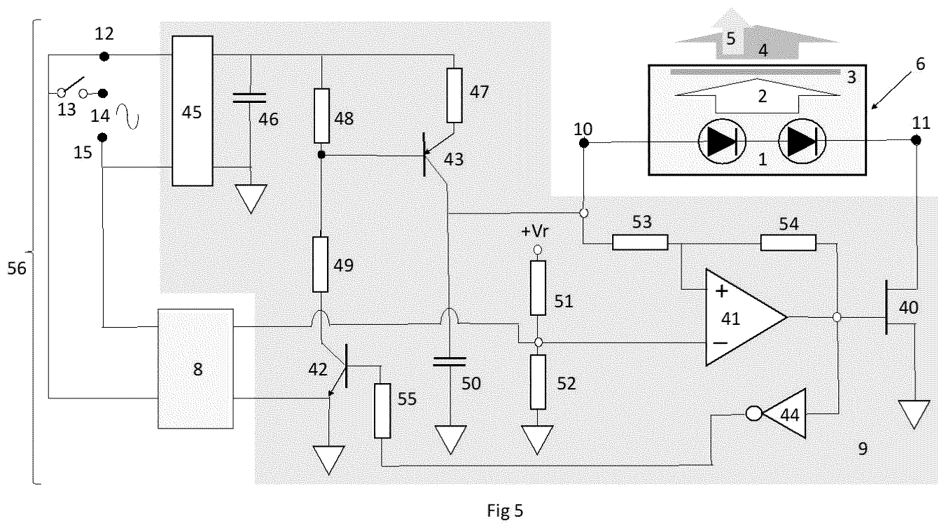

As shown in FIG. 2, for the implementation of a system that allows LED lighting lamps to be given sanitizing capacity, we designed two options for the voltage overexcitation module, the first one is what we call constant voltage overexcitation (57) operates in a similar way to the circuit presented in FIG. 3, only in this one, it is difficult to see the separation between what constitutes the voltage overexcitation driver (9) and what is the remote sanitization level control (8), the second configuration of the overexcitation system makes use of a relaxation overexcitation module (56) as presented in FIG. 5, in this case, a capacitor (50) will be charged to a level greater than 1.5 times the nominal voltage of the LED emitter, and then discharge it onto said LED emitter (1), generating a voltage pulse through the LED emitter (1) which will gradually decrease until capacitor (50) is practically completely discharged, once this has happened, said capacitor (50) shall be charged again to its original voltage level by the action of PNP transistor (43), the resistor (47) acting as a charge limiting element, and by the control exercised by resistors (48) and (49) on the base of the PNP transistor (43), this is possible when the NPN transistor (42) is in saturation, and this NPN transistor (42) will remain in saturation by the action of resistor (55) which in turn is connected to the output of inverter (44), the input of this inverter is in turn connected to transistor (40) gate so that when transistor (40) is in conduction, the NPN transistor (42) will be cut-off, the transistor (40) gate is controlled by comparator (41), which compares the reference level at the junction of resistors (51) and (52) with the voltage level at capacitor (50) affected by the voltage divider formed by resistors (53) and (54), When this level reaches a pre-established upper limit, the output of the comparator (41) will go from a zero level to a voltage that will allow the full conduction of transistor (40) and because resistor (54) is connected between the non-inverting input of the comparator and the output of the comparator, a positive feedback effect is produced that favors the voltage transition at the input of transistor (40) gate, this rapidly discharges the capacitor (50) through the LED emitter (1), so that when the voltage through capacitor (50) has decreased to such a level that the voltage at the non-inverting input of comparator (41) is less than the voltage at the inverting input of the comparator, the comparator (41) will change state at its output and again a positive feedback effect will be created through resistor (54), completely turning off transistor (40) and in this way this cycle will be repeated indefinitely but always with a synchrony between the capacitor's charging period and the capacitor's discharge period, which is achieved by the signal at the transistor (40) gate that is connected to an inverter (44) and then to the NPN transistor (42) that controls the capacitor's charging process, to prevent the LED emitter (1) from burning out, a calculation is made based on the nominal power of the LED emitter (1), the value of capacitor (50), the maximum voltage at which the capacitor is charged and the intrinsic time delays of inverter (44), the NPN transistor (42), the PNP transistor (43) and the comparator (41) itself, this allows determining the voltage and duration of each instantaneous pulse, which are repeated “n” times per second and for which their energy must be multiplied by a factor “n” to evaluate the total energy per second to keep the operation of the LEDs within safe limits, the process to determine the values of each component that guarantee a safe and controlled operation of the LED emitters, can be done empirically, if you do not have at hand all the active and passive elements characteristics that intervene in the time delay between when an action is ordered and it is fully executed, and if you want to have fewer pulses per second, you can place a capacitor with a value in the range of nanofarads between the base of the NPN transistor (42) and ground. In summary, the values of the passive and active components that make up this voltage overexcitation driver are easily estimated so that, even if the LED emitters are powered with voltage pulses that exceed their nominal operating value, this does not cause the destruction of the LED emitter since the nominal total power per second is not exceeded at any time. Also in the FIG. 5 it can be seen that a remote sanitization level control (8) can be connected to the overexcitation driver in such a way that a control output of the remote sanitization level control (8) is connected directly to the inverter input of comparator (41), a rectifier/regulator circuit (45) and a capacitor (46) connected to it, gives a direct current voltage for the circuit operation.

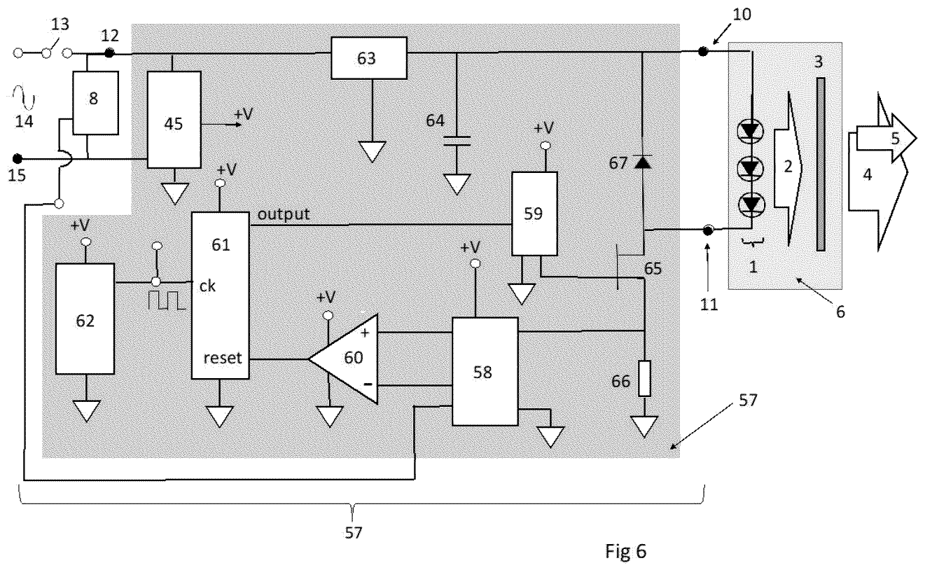

Although FIG. 3 describes a complete white lighting circuit for COVID sanitization, this circuit being completely specific to the use of certain integrated circuits, FIG. 6 presents a more general block diagram of a constant voltage overexcitation module (57), connected to a fluorescent white light LED illuminator (6). FIG. 6 details a circuit in which a capacitor (64) is charged at a voltage greater than 1.5 times the nominal operating voltage of a fluorescent white light LED illuminator (6), this is achieved through a voltage regulator (63) while a rectifier/regulator circuit (45) provides a voltage level for the power supply of the integrated circuits and electronics in general, a current amplifier (59) or MOS driver, controls the activation or cut-off of transistor (65) in such a way that when the output of the multivibrator circuit (61) is at a level one, this level will be presented to the input of current amplifier (59) causing the output of the current amplifier to rise and as it is connected to transistor (65) gate, it is drived into a complete conduction presenting through terminals 10 and 11 of the fluorescence white light LED illuminator (6) all the voltage accumulated in capacitor (64) which has a value between 500 and 2000 microfarads, all this voltage is applied to the blue LED emitter (1) that is inside the fluorescence white light LED illuminator (6), this action consequently causes a small but abrupt increase in voltage through the monitoring resistor (66) which has a value between 1 ohm and one tenth of an ohm, the voltage through this monitoring resistor (66) is connected to the energy control (58) circuit which causes the output of comparator (60) to signal a reset on the multivibrator circuit (61) sending its output to zero and consequently, the current amplifier (59) shuts down transistor (65). When according to pre-established criteria, the power control (58) determines that the voltage through the monitoring resistor (66) has reached a level that could lead to the destruction of the LED emitter (1), an empirical process allows the adjustment and calibration of this energy control (58) so that the sum of all the partial energies applied to the LED emitter (1) in each pulse and multiplied by the number of pulses generated in one second, do not exceed the limit established by the nominal power of the blue light LED emitter (1) inside the fluorescent white light LED illuminator (6), this action is complemented by the adjustment of the oscillator (62), which determines how many overvoltage pulses will be applied per second to the LED emitter (1). The energy control (58) receives a signal from the sanitization level remote control (8) to allow varying the level of the COVID sanitization energy.

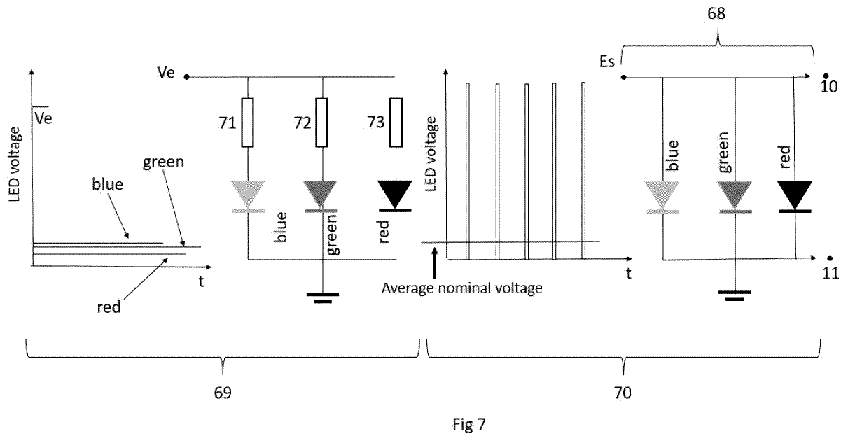

The anti-coronavirus lighting system can be made as already described, using the voltage overexcitation module and an RGB type white light LED emitter, in this case, the RGB type white light illuminator is a unit that contains three LEDs of different colors, red, green and blue whose english acronym gives it the RGB name, as can be seen in the FIG. 7, an RGB type white light illuminator (68) is usually powered using resistors of different values which helps to compensate for the fact that each of the LEDs of different colors have a different nominal operating voltage, for example the red LED can have an operating voltage of 2 volts while the green one normally operates at 2.3 volts and the blue at 2.5 volts, in this way an operation of the three LEDs in parallel does not seem possible with conventional means, as can be seen in FIG. 7, the conventional excitation of RGB LEDs (69) is performed by taking the cathodes that are usually attached in the device and grounding them, while three resistors (71), (72), (73), of different values are placed on the anodes of each of the LEDs and then join the resistors at a single point that is connected to a constant voltage “Ve”, the graph attached to this diagram of the FIG. 7, shows that in this way although the assembly is powered with a general voltage “Ve”, each of the LEDs will be operating at its nominal operating voltage, FIG. 7 also shows the driving layout of an RGB using overvoltage pulses (70), in this diagram the voltage overexcitation module is not shown but the output of this(Es) and all the anodes of the LEDs are joined together as well as each one of the cathodes respectively, as can be seen in the attached graph, in this case, these LEDs connected in parallel, are powered with pulses of short duration but with a voltage that exceeds several times the nominal operating voltage of each one of the LEDs, using this technique, it can be seen that if the RGB set contains LEDs whose nominal operating voltage differential ranges from 2 to 2.5 volts, when powered by narrow pulses of 60 volts, the differential of 0.5 volts goes into the background and the excitation of the LEDs by means of overvoltage pulses produces secondary emission in wavelengths different from the original wavelengths of each LED, in fact, the green LED will emit in the band corresponding to the green but also smaller amounts of light in the blue region, while red will emit essentially light in the red fundamental wavelength, but also smaller amounts of light energy in the rest of the visible spectrum, this in turn reinforces the total emission of blue light, since when an LED is excited with overvoltage pulses, this LED emits light mainly in its natural wavelength but also in lower wavelengths, so the blue LED will emit as a secondary effect light in wavelengths shorter than its original one, the green will emit secondary light in wavelengths lower than its natural plus the fundamental, that is, in the green, blue and violet, while the red LED will emit light in the red band, an also lower levels of yellow, green, blue and violet light.

In summary, the device to give LED lighting lamps capacity for coronavirus destruction, is composed of a voltage overexcitation module that when connected to an LED white light illuminator, either RGB or fluorescence, is given, in addition to its white lighting capacity, the possibility of having an anti-coronavirus sanitizing effect, and the luminaires made in this way can be used in the presence of human beings without having side effects on materials or living beings, particularly working based on the interaction of very narrow pulses of overvoltage on ordinary white light LED illuminators unlike other systems, the use of overexcitation on generic white light LED illuminators by means of overvoltage pulses in the modality of relaxation, optimizes the emission of white light, which also incorporates high-intensity pulses of between 400 and 460 nm in a practically imperceptible way.

What is claimed is:

1. A device to give LED lighting lamps capacity for coronavirus destruction characterized by a voltage overexcitation module that has four terminals, two of which are connected to the electrical power supply through a switch and two others that are connected directly to a fluorescent or RGB white light LED illuminator, where said voltage overexcitation module is in turn constituted by an overexcitation driver and a remote control of sanitization level and where the overexcitation module provides the white light LED illuminators with controlled voltage pulses that exceed by at least 1.5 times the magnitude of the nominal operating voltage of the LED illuminator.

2. The device to give LED lighting lamps capacity for coronavirus destruction, in accordance with claim number 1, where the voltage overexcitation driver is a relaxation-type circuit, characterized by having a capacitor that is charged through a transistor that constitutes a current source controlled by a second transistor and the corresponding polarization and reference resistances, the latter transistor activates or cuts the current supply to the capacitor based on a signal that in turn controls the activation or cut-off of a field effect transistor that connects to the LEDs of the illuminator in such a way that when this transistor is activated, the entire charge of the capacitor is drained through the LEDs, the field-effect transistor gate is controlled by a comparator with positive feedback that compares the voltage in the capacitor with a reference voltage level set by a voltage divider connected to a fixed voltage so that the discharge of the capacitor through the LEDs is synchronized with the capacitor charge, so that while the capacitor load is being carried out, it is not possible for the capacitor to be discharged and while the capacitor is discharging, the charge of the same capacitor is suspended, all these circuits being powered by a rectifier and a power capacitor, while a sanitization level control circuit regulates the maximum level of the voltage of the discharge pulses by modifying the reference level to the inverter input of the comparator, this level being modifiable by a sequential action of the switch by means of which the voltage overexcitation module is connected to the electrical power supply.

3. The device for give LED lighting lamps capacity for coronavirus destruction in accordance with claim 1 where the remote control of the sanitization level is characterized by the fact that it is constituted by a multivibrator connected in the form of a bistable unit type FLIP-FLOP where its reset input is connected to a capacitor that in turn is connected to the positive supply voltage of the circuit and on its other side grounded by means of a resistor, the inverter output of the multivibrator is connected to the input data and in turn connected to the gate of a field-effect transistor whose source is connected to earth and whose drain is connected to a resistor whose free end is connected to an element of the overexcitation driver circuit that controls the energy of each pulse applied to the LEDs, the clock input of the multivibrator is connected to a voltage divider which has one end grounded and the other end connected to the point where a grounded capacitor and a diode are connected to the electrical power supply and connected in such a way that the clock input receives signals within the operating range of the multivibrator but in such a way that when the electrical power supply is suspended, the voltage in the capacitor drops until it crosses the operating limit equivalent to zero at the multivibrator clock input.

4. The device for give LED lighting lamps capacity for coronavirus destruction in accordance with claim 1 where the voltage overexcitation driver is characterized by being a device that feeds the LED unit by means of constant voltage pulses with a magnitude that exceeds more than 1.5 times the nominal operating voltage of the LEDs and that has a capacitor which is charged by a voltage regulator device to a value greater than 1.5 times the nominal operating voltage of the LEDs or of the LED array to whose positive input the capacitor is connected, while the cathode or negative input of the white LED light illuminator is connected to an electronic switch consisting of a field-effect transistor whose source is grounded by a monitoring resistor while the junction between the transistor and the resistor are connected to an energy control circuit which in turn is connected to a comparator whose output is connected to the reset input of a multivibrator whose output is connected to a MOS driver or current amplifier, the output of which controls the activation or cut-off of the transistor to whose gate it is connected, an oscillator is connected to the multivibrator's clock input determining the frequency of the pulses emitted, while the energy control circuit dynamically determines the duration of each pulse.

5. The device for give LED lighting lamps capacity for coronavirus destruction in accordance with claim 1 whereby the voltage overexcitation module consists of a voltage overexcitation driver that feeds the white light LED illuminator, with pulses of constant voltage and amplitude greater than 1.5 times the nominal operating voltage of the LED or set of LEDs inside of the white light LED illuminator, being this a self-oscillating driver which is powered by a regulator circuit that provides the integrated circuits with the level of direct current voltage necessary for their operation by taking their energy from a rectifier and a capacitor connected to the electrical power supply through an external switch which has the double function of allowing the activation of the white light LED illuminator and at the same time, controlling the proportion of blue light pulses between 400 to 460 nm with respect to the total light generated by the illuminator including white light by a sequential action of this switch, this capacitor remains charged at a voltage greater than 1.5 times the nominal operating voltage of the LED or LEDs array and connected to the anode or positive input of the LED or LEDs array, while the cathode or negative input of said LED or set of LEDs, is connected to a field effect transistor whose gate is connected to the output of an exciter type current amplifier or MOS driver such as the MCP14A0602 whose input is connected to the supply voltage of this integrated circuit while the activation input of the same (EBL) is connected to the positive voltage supply of the MOS driver by means of resistor, and connected to a capacitor that is itself grounded in such a way that when the voltage through this capacitor exceeds the threshold level of the current amplifier or MOS driver, the output of this one will go to a level one, and when this level is below the threshold level it will go to a zero level, allowing the activation and cutting of the field effect transistor whose source is grounded by a monitoring resistor which in turn is connected to the base of an NPN transistor by a resistor, being that the emitter of the NPN transistor is connected with its grounded emitter and its collector to the capacitor that is connected to the control input of the current amplifier or MOS driver, when the voltage across the monitoring resistor exceeds the bias threshold of the NPN transistor (0.7 Volts), this transistor will go into a conduction state by discharging the capacitor connected to its collector causing the field-effect transistor to stop passing current abruptly and remaining so until the capacitor connected to the control input of the current amplifier is charged through the resistor connected between this point and the supply voltage and when this happens the cycle is repeated for the generation of a new voltage pulse on the LED or LEDs array, the amount of energy delivered to each pulse is determined by the action of a resistor connected to the base of the NPN transistor and to a grounded FET transistor that when the latter is in full conduction, the aforementioned resistor forms a voltage divider together with the resistor connected between the base of the NPN transistor and the monitoring resistor, which causes the voltage on the monitoring resistor to be much greater than 0.7 volts to activate the NPN transistor and suspend the flow of energy through the LED or set of LEDs, the FET transistor is connected to the inverter output of a CD4013 type multivibrator and to the data input so that every time the clock input of this multivibrator changes state, so will the non-inverting output of the multivibrator, the reset input is connected to ground by a resistor and to the supply voltage of the circuit by a capacitor while the clock input is connected to a voltage divider connected to earth and to a capacitor connected by a diode to the alternating current electrical power supply when the other end of said capacitor is connected to the ground, this arrangement limits the input voltage level in the clock (CK) of the multivibrator and allows the control of state changes of the outputs of the multivibrator based on a sequence of connections and disconnections of the external wall switch that controls the supply of electrical power to all circuits.
